(19) United States
(12) Patent Application Publication
(10) Pub. No.: US 2023/0212558 A1
(43) Pub. Date: Jul. 6, 2023

(54) CONVERSION OF STRAND DISPLACEMENT APTAMERS INTO MOLECULAR BEACONS

(71) Applicant: Palo Alto Research Center Incorporated, Palo Alto, CA (US)

(72) Inventors: Jerome UNIDAD, San Francisco, CA (US); Sean DORIS, San Francisco, CA (US)

(73) Assignee: Palo Alto Research Center Incorporated, Palo Alto, CA (US)

(21) Appl. No.: 17/570,073

(22) Filed: Jan. 6, 2022

(51) Int. Cl. C12N 15/10; C12N 15/115; G16B 35/00; G01N 15/10

(52) U.S. Cl. CPC C12N 15/1089; C12N 15/115; G16B 35/00; G01N 15/1056; C12N 2310/16; C12N 2320/13; G01N 2015/1006

(57) ABSTRACT

Molecular beacons and developmental methods related thereto. Methods include obtaining a nucleotide sequence for an aptamer that binds to a target analyte. The aptamer comprises a binding domain nucleotide sequence, a first domain nucleotide sequence, and a displacement domain nucleotide sequence complementary to the first domain nucleotide sequence. A molecular beacon is developed based on the nucleotide sequence of the aptamer by preserving the binding domain nucleotide sequence and truncating or extending one or both of the first domain nucleotide sequence or the displacement domain nucleotide sequence. The resultant molecular beacon is developed such that the molecular beacon comprises a Gibbs free energy value that is greater than the Gibbs free energy value of the aptamer.

### SEQUENCE LISTING

The Sequence Listing created on Mar. 23, 2022 and submitted in text format (.txt) on Mar. 24, 2022, named ‘Sequence Listing_078313-000056 (2).txt’, (431 bytes), is incorporated herein by reference in its entirety.

### BACKGROUND

The present disclosure relates to converting strand displacement aptamers into molecular beacons.

The U.S. and broader international scientific community continue to lack a cost-effective and reliable technique for converting strand displacement aptamers into aptamer-based molecular beacons for surface tethering and continuous sensing operation that preserves the binding specificity and affinity of the original strand displacement aptamers.

Aptamers are relatively small oligonucleotide or peptide molecules of generally less than about 100 nucleotides that are capable of binding to target biological material (analytes) with high affinity and specificity. An aptamer may be capable of distinguishing between biological materials differing by only several atoms. A vast array of biological materials or compounds may be targeted by aptamers, including, but not limited to, complex tissues, cells, proteins, peptides, metabolites, pharmaceuticals, biologics and other small molecules. Accordingly, aptamers have proven valuable in various technologies including, but not limited to, biological research, diagnostic, therapeutic, and theranostic applications ranging from biomarker identification, drug discovery, protein expression control, and targeted delivery of therapeutic agents. More recently, aptamers have found interest in fluorescence or colorimetric bioassays, as well as in biosensors for continuous monitoring of target analytes.

Strand displacement aptamers are composed of double-stranded deoxyribonucleic acid (DNA) and can be converted into single-stranded aptamers for use as molecular beacons. Strand displacement aptamers structurally switch between a DNA/DNA hybridized configuration (double-stranded) to a DNA/target analyte configuration (binding a target biological analyte of interest), exhibiting distinct and different structural configurations between the two states (also referred to herein as the target-unbound configuration and the target-bound configuration, respectively). In switching from a DNA/DNA to DNA/target analyte configuration, the non-target bound DNA strand (referred to herein as the “displacement strand”) is un-hybridized from the remaining DNA strand (referred to herein as the “main strand”) and goes elsewhere in the solution; that is, the displacement strand disassociates from the main strand. It follows that this switch from the DNA/DNA to the DNA/target analyte configuration also reflects a drastic change in aptamer configuration, including, for example, physical dimensions, spatial configuration, molecular conformation, and the like, and any combination thereof. On the other hand, molecular beacons can also structurally switch between an unbound configuration (DNA or RNA strand only) to a target analyte bound configuration (DNA/target analyte) such that the configuration (e.g., physical dimensions, spatial configuration or molecular conformation, and the like, and any combination thereof, as mentioned above) of the molecular beacon between target-bound and target-unbound states are sufficiently different to detect (e.g., optically, electrochemically, or through other means) which state the molecular beacon is in. The key difference between a strand displacement aptamer and a molecular beacon is that the former exists initially as two or more molecules of DNA that are at least partially hybridized when not target-bound whereas the latter is always a single molecule of DNA.

Traditional methods for selecting aptamers for binding to a particular target biological analyte focus on binding capability and specificity rather than structure-switching, and the resultant molecular beacons are often limited to certain un-grafted or free-in-solution assaying or sensing uses (e.g., fluorescence-based sensing). Yet, strand displacement, structure-switching aptamers—converted into “molecular beacons”—are compatible for additional uses. For example, these converted molecular beacons can be employed for continuous, reagentless biorecognition sensing applications, such as when the molecular beacon is bound to a surface without the need for a reagent to regenerate sensing capability and, thus without the potential of said reagent to interfere with the reaction equilibrium of the molecular beacon.

Aptamer-based biosensors have been demonstrated in the literature using molecular beacons or aptamers that are selected for specific binding, such as for tobramycin (an antibiotic), for thrombin (a blood clotting factor), and for cocaine (an illegal drug), among others. However, there is not currently a method for converting strand displacement aptamers into molecular beacons suitable for tethered (e.g., electrode-bound), continuous sensing applications, thereby limiting the use of strand-displacement aptamers for use in biosensing a gamut of target analytes (e.g., cortisol and other similar analogues). Moreover, there is no currently known method of converting known strand-displacement aptamers that are specific to known targets in a structure-switching context into a form suitable for continuous, reagentless biosensing without the use of costly and time-consuming screening campaigns or molecular docking exercises. Developing a way to convert pre-selected, high-specificity strand displacement aptamers into molecular beacons provides an avenue to permit the conversion of liquid-based assays into continuous, reagentless biosensing that is more suitable for in vivo and in vitro applications, and further expand the utility of aptamer-based biosensing to analyte targets with pre-existing strand-displacement aptamers.

### SUMMARY OF INVENTION

The present disclosure relates to converting strand displacement aptamers into molecular beacons.

A nonlimiting example of the present disclosure includes a method comprising obtaining a nucleotide sequence for an aptamer that binds to a target analyte, the aptamer having a binding domain nucleotide sequence, a first domain nucleotide sequence, and a displacement domain nucleotide sequence complementary to the first domain nucleotide sequence; determining a first Gibbs free energy value for the aptamer; and developing a molecular beacon based on the nucleotide sequence of the aptamer by preserving the binding domain nucleotide sequence and truncating or extending one or both of the first domain nucleotide sequence or the displacement domain nucleotide sequence, the truncating or extending performed such that the molecular beacon comprises a second Gibbs free energy value that is greater than the first Gibbs free energy value of the aptamer.

A nonlimiting example of the present disclosure includes a molecular beacon comprising a portion of a nucleotide sequence of an aptamer that binds to a target analyte and has a first Gibbs free energy value, the portion of the nucleotide sequence forming the molecular beacon comprising: a preserved binding domain nucleotide sequence of the aptamer, wherein the binding domain sequence of the aptamer binds to the target analyte; and a truncated or extended one or both of a first domain nucleotide sequence of the aptamer or a displacement domain nucleotide sequence of the aptamer, wherein the truncation or extension is performed to develop the molecular beacon comprising a second Gibbs free energy value that is greater than the first Gibbs free energy value of the aptamer.

### BRIEF DESCRIPTION OF THE DRAWINGS

The following figures are included to illustrate certain aspects of the disclosure, and should not be viewed as exclusive configurations. The subject matter disclosed is capable of considerable modifications, alterations, combinations, and equivalents in form and function, as will occur to those skilled in the art and having the benefit of this disclosure.

FIG. 1A depicts a conventional, illustrative strand displacement aptamer.

FIG. 1B depicts an illustrative molecular beacon having been converted from the illustrative aptamer of FIG. 1A, according to one or more methods of the present disclosure.

FIG. 2A depicts the nucleotide sequence of a conventional cortisol strand displacement aptamer, including the displacement strand and the main strand.

FIG. 2B depicts the nucleotide sequence of a conventional cortisol strand displacement aptamer, including only the main strand, the displacement strand having been displaced.

FIGS. 3A and 3B depict the converted nucleotide sequence of the conventional cortisol strand displacement of FIG. 2B into a molecular beacon candidate, according to one or more methods of the present disclosure, but failing to meet the required Constraints described herein.

FIG. 4 depicts the converted nucleotide sequence of the conventional cortisol strand displacement of FIG. 2B into a molecular beacon candidate, according to one or more methods of the present disclosure, and meeting the required Constraints described herein.

FIG. 5 depicts the converted nucleotide sequence of the conventional cortisol strand displacement of FIG. 2B into a molecular beacon candidate, according to one or more methods of the present disclosure, and not meeting the required Constraints described herein.

### DETAILED DESCRIPTION

The present disclosure relates to converting strand displacement aptamers into molecular beacons.

More particularly, the present disclosure relates to strand displacement aptamers that can be converted and optimized as molecular beacons that structurally switch between states upon binding target biological analytes, such as for use in the medical, food, biotechnology, environmental, or related industries. The molecular beacons described herein may be used in biorecognition sensing technologies, such as electrochemical sensing and are able to be tethered to a surface, such as an electrode, for continuous operation as a sensor without the need for a reagent. That is, the molecular beacons described herein are able to operate over a period of time rather than merely function on a single use (e.g., assay) basis.

Moreover, the strand displacement aptamers of the present disclosure can be pre-selected, having known binding to particular target analytes, and converted to a suitable molecular beacon form purely in silico, thereby avoiding costly and time-consuming screening campaigns (e.g., using SELEX) or molecular docking simulations that are currently run whenever one needs to select an aptamer for a new target. Further, the strand displacement conversion methods described herein are optimized to maintain the binding specificity of the original aptamer in the converted molecular beacon form while ensuring suitability for surface tethering at one or more points and continuous sensing operation based on target-bound v. target-unbound configurations, and thus can be used as continuous sensors. The utility of the converted molecular beacon form in continuous, reagentless biosensing, such as electrochemical sensing, relies on the drastic structure switch (e.g., change in physical dimension, spatial configuration, molecular conformation, and the like, and any combination thereof) of the molecular beacon when target-bound v. target-unbound.

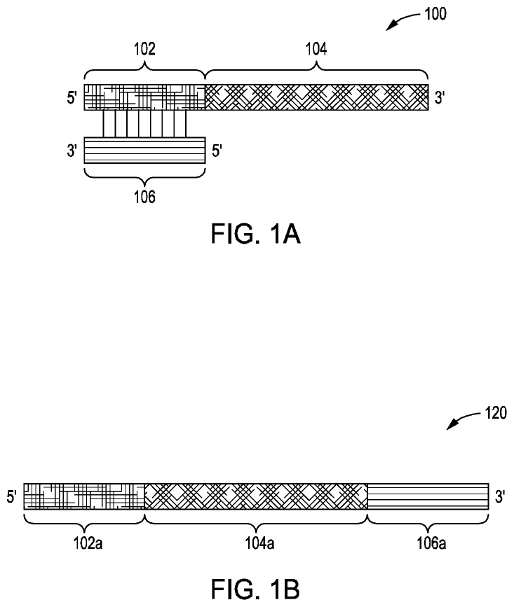

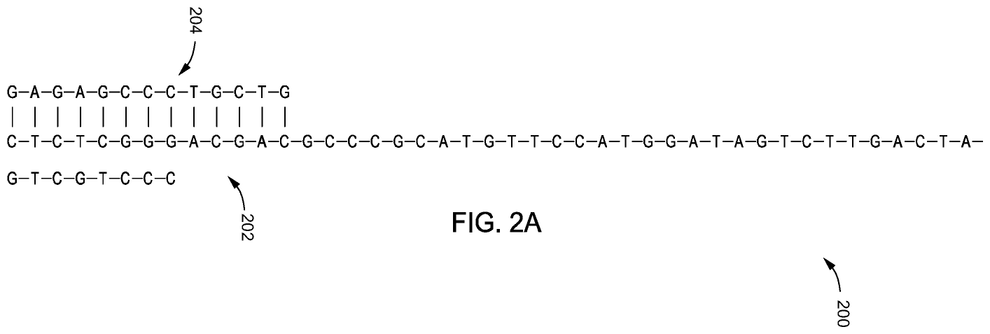

The present disclosure provides a method for converting a known strand displacement aptamer having two or more DNA strands (typically two strands) and with affinity to a known or reasonably inferred target biological material into a molecular beacon. Referring to FIG. 1A, an illustrative strand displacement aptamer 100 is depicted. As shown, the aptamer 100 is composed of a double stranded DNA having 5′ and 3′ ends. More particularly, the aptamer 100 comprises a first domain (the “main strand”) composed of DNA sections 102 and 104, where 104 includes the binding domain specific to a particular target analyte. A second strand (the “displacement strand”) composed of complementary DNA section 106 is bound to DNA section 102 and serves as a displacement domain, such that when aptamer 100 encounters its target analyte, DNA 106 is displaced and no longer bound to DNA 102, 104. That is, when the aptamer encounters its target analyte, the displacement strand is displaced and no longer bound to the main strand.

Referring now to FIG. 1B, illustrated is molecular beacon 120 having been converted from aptamer 100 of FIG. 1A, according to one or more methods of the present disclosure. As shown, the molecular beacon 120 comprises each of DNA 102, 104, and 106 from FIG. 1A, labeled 102a, 104a, and 106a, respectively. Molecular beacon 120 is a single strand of DNA, rather than the strand displacement double stranded aptamer 100 of FIG. 1A. In practice, the molecular beacon 120 may be surface tethered by either the 5′ end or the 3′ end. In either case, the target binding region, DNA 104a, is available for binding the target analyte. The sequence of DNA 104a is also preserved from the original sequence of DNA 104.

It is to be appreciated that while FIG. 1B shows that 102a and 106a are identical or substantially identical to 102 and 106 of FIG. 1A, in alternative aspects, DNA 102a and 106a may be selected from a library of known complementary DNA pairs that are suitable given the specific experimental conditions. Such experimental conditions may include, but are not limited to, attachment chemistry; operational parameters of the resultant sensor, such as temperature, pH, applied voltage, solvent quality; and the like; and any combination thereof. In one or more aspects of the present disclosure, it is envisioned that a library of such complementary DNA pairs could be well-known and pre-selected based on their performance in previously developed sensors or assays. Any selected DNA 102a and 106a should be at least partially complementary, including fully complementary, and should not compete with the target binding region 104a for binding a target analyte. As used herein, the term “partially complementary,” and grammatical variants thereof, refers to a sequence complementarity, based on Watson-Crick pairing, that is fulfilled in the range of about 60% to about 95%, encompassing any value or subset therebetween, including whether the strands are of the same or different lengths. That is, the sequence complementarity, whether the same length or otherwise, of DNA 102a and 106a is between about 60% to about 95%.

Converting aptamer 100 into molecular beacon 120 proceeds under at least four constraints. First, it is imperative that the target-binding region 104 of aptamer 100 is preserved. That is, DNA 104 of aptamer 100 and DNA 104a of molecular beacon 120 are identical or at least substantially identical to ensure that its affinity and specificity to a target analyte of interest are maintained, as shown in below as Constraint 1 (and with reference to numerical identifiers in FIGS. 1A and 1B). As used herein, the term “substantially identical,” with reference to the DNA sequence preservation of a target binding region of an aptamer (e.g., DNA 104) and target binding region of a converted molecular beacon (e.g., DNA 104a), means at least one or both of the following: the base pair deviation between the target binding region of the aptamer (DNA 104) and the target binding region of the molecular beacon (DNA 104a) is equal to or less than about 20% and the number of possible secondary structures for both the target binding region of the aptamer (DNA 104) and target binding region of the molecular beacon (DNA 104a) (e.g., solvable by structure predicting platforms, such as mFold or RNAFold) do not differ by greater than about 15%.

104=/≅104a

### Constraint 1

Second, the lowest energy (Gibbs free energy, ΔG) secondary structure for molecular beacon 120 must have a ΔG equal to or greater than the ΔG of the lowest energy secondary structure of aptamer 100 when displacement DNA 106 is no longer bound, as shown below as Constraint 2 (and with reference to numerical identifiers in FIGS. 1A and 1B). This ensures that the target biological analyte will show greater affinity to the molecular beacon 120 configuration (promoting displacement of the displacement strand 106a) and that the molecular beacon is sufficiently unstable to favor target binding compared to the original aptamer 100.

ΔG(104+102)≤ΔG(104a+102a+106a)

### Constraint 2

As provided in the present disclosure, this Constraint 2 may be manipulated by extending or truncating either or both of 102a or 106a, for example, to ensure that the molecular beacon 120 has a desired Gibbs free energy while ensuring that substantial Watson-Crick pairing is maintained, as described above. Note that the sequence length of 102a and 106a may different, so long as sufficient Watson-Crick pairing is maintained (reflected by relevant secondary structure predictions) and may be done to satisfy Constraint 2.

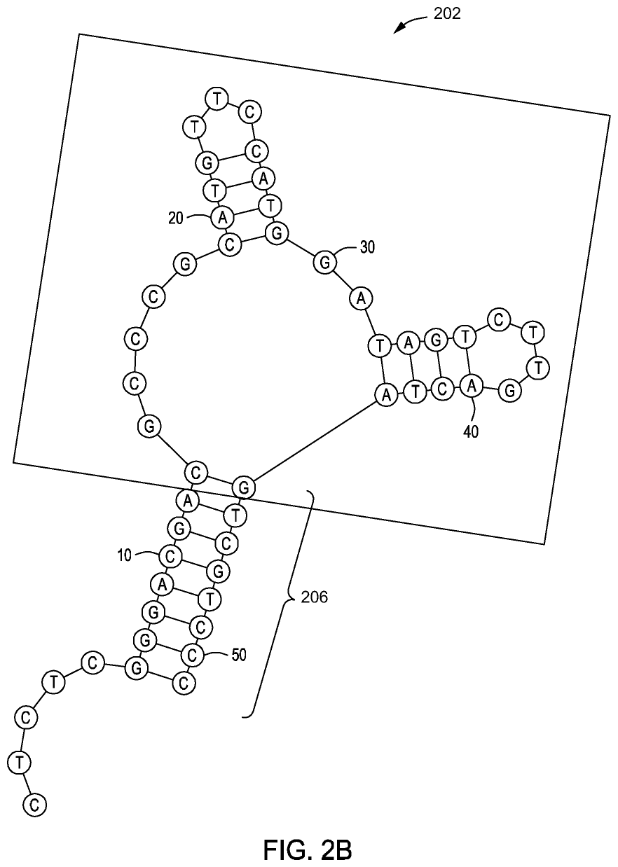

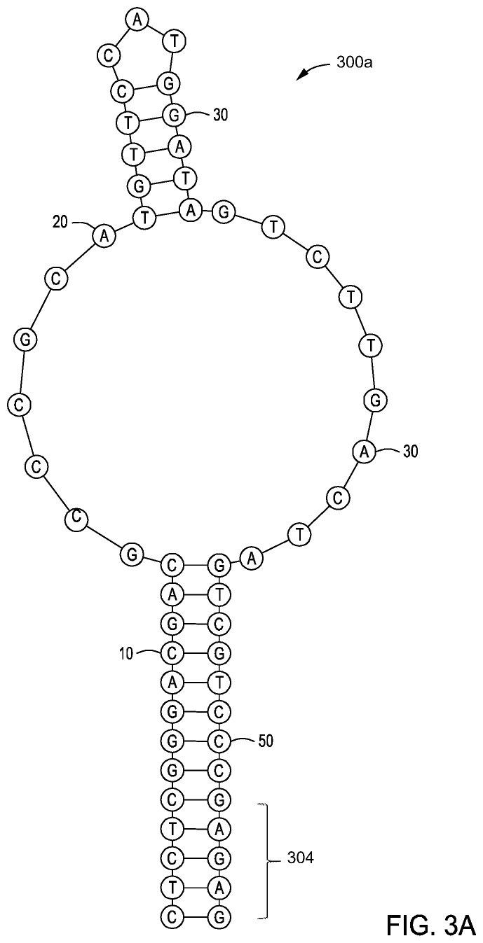

Third, for a candidate molecular beacon sequence DNA 104a+DNA 102a (mathematical sum), the lowest ΔG secondary structure that is predicted or calculated must be substantially similar in terms of Watson-Crick paired nucleotides (i.e., about 60% to about 95% or more) to the lowest ΔG secondary structure predicted or calculated for the original aptamer sequence DNA 102+DNA 104 (mathematical sum). This similarity in geometry will be made apparent in the subsequent discussion of FIGS. 2B, 4, and 5. Base pair mismatches (i.e., each differently paired or unpaired nucleotide counts) should not differ by greater than about 15% of the total number of nucleotides of the original aptamer.

Lastly, conversion of aptamer 100 into molecular beacon 120 should proceed by maximizing the difference between the apparent electron transfer rates (kapp) of the target bound and target unbound molecular beacon 120. The kapp of the molecular beacons described herein may be estimated in silico using published experimental data, by simulation using stochastic Langevin dynamics to calculate the Gibbs free energy landscape given variable electrostatic barrier heights based on the span length of DNA 102a and 106a, by chain length arguments using polymer physics (statics and dynamics), by molecular docking and/or by other molecular simulations (e.g., ab initio, coarse-grained, and the like) that can be performed to qualitatively estimate the difference in kapp for the target-bound and target-unbound states. It is to be appreciated that this is a “soft” constraint and is primarily used to ensure the likelihood that the molecular beacon 120 is useful in a biosensing application; that is, that the resulting molecular beacon 120 has sufficiently distinguishable kapp between the target-bound and target-unbound states. It is further to be appreciated that the specificity of the aptamer is primarily dictated by Constraint 1 and, thus should not be affected or substantially affected by enforcing this Constraint 4 (nor Constraint 2 or Constraint 3). Accordingly, enforcing this Constraint 4 may result in the modification of strands 102a and 106a in a manner that does not violate the previous three constraints.

Accordingly, a first apparent electron rate value (k1) representative of the molecular beacon being bound to the target analyte at the preserved binding domain nucleotide sequence and a second apparent electron rate value (k2) representative of the molecular beacon being unbound to the target analyte at the preserved binding domain nucleotide sequence, and the molecular beacon being truncated or extended such that the difference between the first apparent electron rate value and the second electron rate value is maximized. In one or more aspects, the ratio of k1/k2 or k2/k1 is in the range of 2 to 1000, such as in the range of about 2 to about 500, or about 500 to about 1000, encompassing any value and subset therebetween.

In one or more aspects, the molecular beacon 120 may be tethered at the 5′ or 3′ end to a surface or substrate (e.g., an electrode) and a redox active center attached to the non-tethered end to functionalize the molecular beacon. Examples of suitable redox active centers may include, but are not limited to, a metallocene (e.g., ferrocene, ruthenocene, osmocene), methylene blue, an anthraquinone, a benzoquinone, a napthoquinone, a viologen (e.g., methyl viologen, benzyl viologen, long chain alkyl viologen), nile blue, and the like, derivatives thereof (e.g., Atto MB2), and any combination thereof. In such cases, the molecular beacon 120 may be utilized as a sensor, such as an electrochemical-based sensor (e.g., detectable voltametrically or amperometrically) or other sensing modality-based sensor requiring surface tethering of the molecular beacon for immobilization. When used as a sensor, the aptamer-based molecular beacon serves the role of a sufficiently sensitive picomolar binding element and where the resulting sensor has a sensitivity down to the nanomolar and/or picomolar ranges. Such other sensing modalities may include, but are not limited to, fluorescence (quenching and non-quenching), including florescence resonance energy transfer; surface-enhanced raman spectroscopy; surface enhanced infrared absorption; optical sensing (e.g., absorption spectroscopy, colorimetry, dye displacement and the like); coulometric sensing; thermal sensing; gravimetric sensing; calorimetric sensing; and the like; and any combination thereof. Moreover, the molecular beacons described herein are not limited to use in biological sensing applications, but may further be used in traditional aptamer applications in which specific target binding is needed and where a single-stranded aptamer in the form of a molecular beacon is advantageous, such as targeted drug delivery, analytical separations, biomarker imaging, theranostics (e.g., controlled release of drug payloads), and the like, and any combination thereof, without departing from the scope of the present disclosure.

While the various strategies for transducing aptamers into molecular beacons in accordance with the present disclosure are described with reference to one or more particular initial aptamers, it is to be understood that any known strand displacement aptamer may be used according to the methods described herein, without limitation.

According to one or more aspects of the present disclosure, existing strand displacement aptamers may be engineered to optimize structure switching functionality into molecular beacons based at least on the above four (4) Constraints. It is necessary that the thermodynamic stability (Gibbs free energy) of the designed molecular beacon(s) be sufficiently tuned such that it favors structure switching in the presence of a target analyte; that is, that the displacement strand (106a of FIG. 1B) is preferentially displaced in the presence of the target analyte. This may be achieved by varying the number of nucleotides forming the displacement strand. Generally, in one or more aspects of the present disclosure, the Gibbs free energy (ΔG) of the produced molecular beacon 120 is at least greater than the ΔG of the original aptamer, as described above; that is, such that ΔG (120)−ΔG (100)>1 kcal/mol. And as discussed above with reference to Constraint 3, the lowest energy secondary structures for both 120 and 100 are sufficiently similar in terms of base pair matching.

Referring now to FIG. 2A, illustrated is the nucleotide sequence of a conventional cortisol strand displacement aptamer 200, including displacement strand 204 (shorter strand of GAGAGCCCTGCTG) and main strand 202 (remaining strand). As shown, the displacement strand 204 and main strand 202 have sequence complementarity between them. The main strand 202 is sufficiently selective against eight (8) out of nine (9) steroids that are closely related to cortisol (hydrocortisone), including deoxycorticosterone 21-glucoside, dehydroisoandrosterone 3-sulfate, deoxycorticosterone, progesterone, dehydroepiandrosterone, 25-hydroxycholesterol, 7α,25-dihydroxycholesterol corticosterone and aldosterone.

Referring now to FIG. 2B, and with continued reference to FIG. 2A, illustrated is one secondary structure of the main strand 202 of the conventional cortisol strand displacement aptamer 200 of FIG. 2A (nucleotides 1-51). The end sequence pertaining to segment 206 comprises the eight (8) nucleotide sequence GTCGTCCC (referred to as the “switching domain”) and is the segment that switches the conformation of the main strand 202 in the presence the target analyte, cortisol. The analyte (cortisol) binding region that forms the specific binding pocket is shown as numbered nucleotides 13 to 44 (see boxed region). That is, the unbound, loop domain of the main strand 202 represents the analyte binding region (referred to as the “binding domain”). The main strand 202 has a Gibbs free energy of about −14.1 kcal/mol. When bound with the target analyte (cortisol), the main strand 202 becomes more stable, and has a Gibbs free energy that is lower by approximately 14.8 to 16.3 kcal/mol less compared to its unbound state (thus, ΔG being in the range of about −29.2 kcal/mol to about −30.4 kcal/mol). It is to be appreciated that the ΔG=−29.2 to −30.4 kcal/mol achieved in the presence of the target analyte is lower than the free energy of the original cortisol strand displacement aptamer 200 (in its unfolded configuration) hybridized with the displacement strand 204, which has a ΔG=−20.7 kcal/mol, which is lower than ΔG=−14.1 kcal/mol for the main strand 202 structure in FIG. 2B, without the cortisol in the binding pocket. As such, the presence of cortisol stabilizes the secondary loop structure of the molecular beacon 202 shown in FIG. 2B. Without being bound by theory, it is believed that the main strand 202 wraps around the cortisol molecule to form a stable, highly specific binding domain (also referred to herein as a “binding pocket”). It is to be understood that the specific secondary structure of interest (i.e., the topology of the structure of the main strand 202 shown in FIG. 2B) is unique and modified aptamers which are expected to form similar specific binding need to be similar to this structure (i.e. to within about 15% of paired/unpaired nucleotide counts), as mentioned in Constraint 3 above.

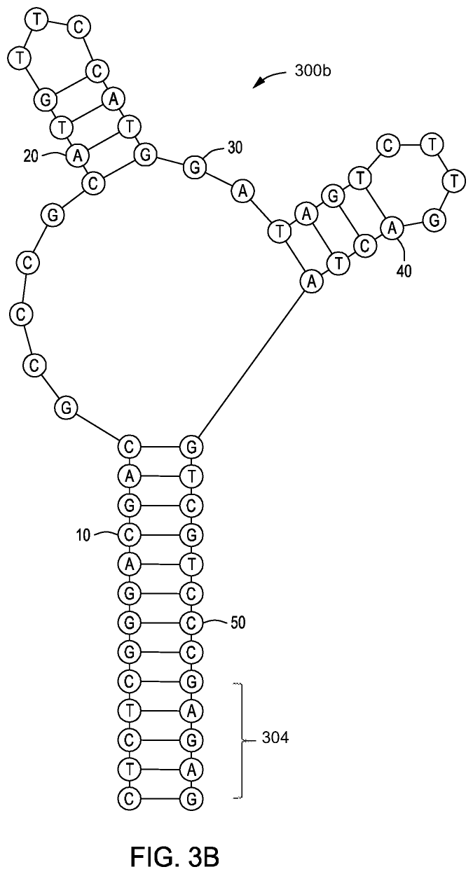

FIGS. 3A and 3B show two lowest energy secondary structures determined from a candidate sequence for a molecular beacon, according to one or more aspects of the present disclosure, based on the template prescribed by FIG. 1B and described herein. Secondary structures 300a and 300b represent alternative secondary structures having the same DNA sequence. This candidate sequence results from directly appending the main strand sequence 202 of FIG. 2A and a portion of the sequence 204 of the original displacement strand as shown in FIG. 2A. The calculated ΔG value for the aptamer secondary structures 300a and 300b, respectively, are −25.8 kcal/mol and −25.2 kcal/mol. Accordingly, the folded aptamer secondary structures 300a and 300b are stable, such as with respect to other secondary structures available (e.g., as shown in FIG. 2B), without requiring the presence of cortisol to stabilize this structure. Hence, the candidate sequence depicted in secondary structures 300a and 300b does not satisfy Constraint 2, despite satisfying Constraint 1—and is thus not an ideal molecular beacon candidate.

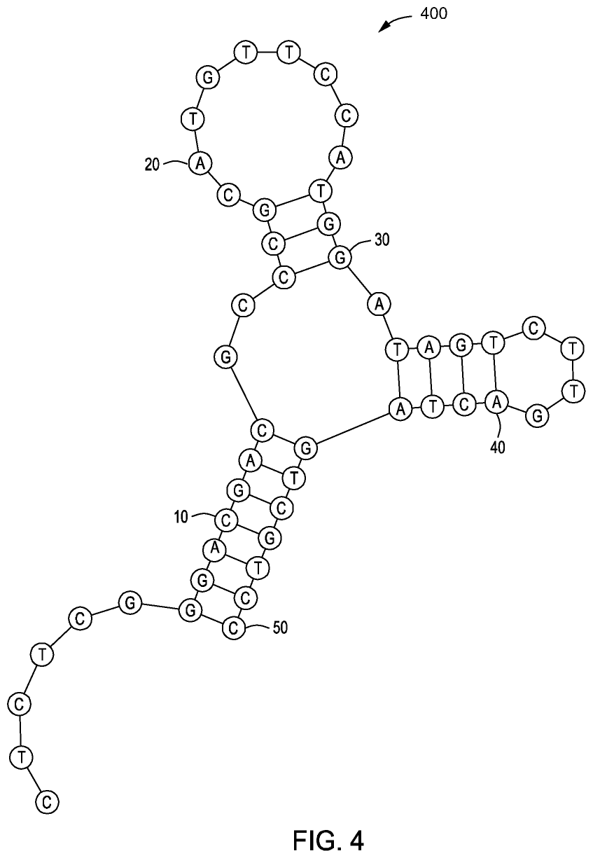

FIG. 4 shows a lowest energy secondary structure, 400, of an aptamer candidate sequence based on the candidate sequence from secondary structures 300a and 300b. The candidate sequence depicted in FIG. 4 is identical to that depicted in FIGS. 3A and 3B with truncated nucleotides 51 through 56, which meets Constraints 1 through 4, thereby being an ideal candidate for a molecular beacon. By truncating the sequence of secondary structures 300a and 300b (removing nucleotides 51 through 56), the free energy of secondary structure 400 is raised to ΔG=−10.9 kcal/mol. It is to be appreciated that this value is larger than the ΔG=−14.1 kcal/mol from the structure in FIG. 2B, which satisfies Constraint 2. Simultaneously, the sequence of the binding domain, represented by nucleotides 13 to 44, is identical to sequence of the binding domain in the original aptamer (FIG. 2A), as well as nucleotides 13 to 44 of main strand aptamer in FIG. 2B, which satisfies Constraint 1. Further, the structure in FIG. 4 is identical to FIG. 2B to within 12.5% of paired/unpaired nucleotide counts, which satisfies Constraint 3.

With continued reference to FIG. 4, and regarding the softer Constraint 4, the shorter span of the aptamer when folded and bound with the target (i.e., the shortest end-to-end distance from nucleotide 1 to 50, which is 8 nucleotides) ensures that it is sufficiently distinguishable from its target-unbound form. The target unbound form is such that the entire aptamer is essentially unfolded, i.e., an end-to-end distance of 50 nucleotides. This ensures that candidate aptamer 400 can be easily distinguished through different sensing modalities, such as electrochemical, due to the drastic shortening of this shortest end-to-end distance and its impact in chain dynamics.

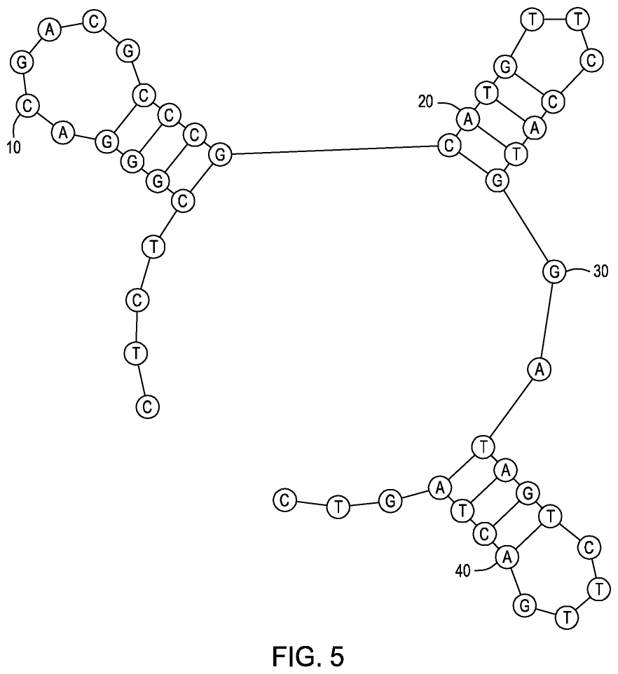

It is to be appreciated that enforcing Constraint 4 can result in significant shortening of the displacement strand for any candidate aptamer in a manner that results in lowest free energy secondary structures that are otherwise in violation of the other Constraints. A counter example that highlights this is provided in FIG. 5, which shows the lowest free energy structure for a candidate aptamer (molecular beacon) where the displacement sequence is shortened too much. This shortening results in stable secondary structures (including that shown in FIG. 5) that are completely different from the secondary structure with optimal target binding pockets such as those in FIG. 2B (original aptamer) or FIG. 4 (optimized molecular beacon candidate). FIG. 5 highlights that this in silico redesign of the aptamer of FIG. 2A, for example, must simultaneously satisfy all four constraints.

Accordingly, in one or more aspects of the present disclosure, a cortisol molecular beacon is provided having the sequence 5′-CTCTCGGGACGACGCCCGCATGTTCCATGGATAGTCTTGACTAGTC-3′ (SEQ ID No. 1).

As provided above, known aptamers that target known analytes may be manipulated as described herein in silico other than cortisol and are not considered to be particularly limiting. Specific target analytes may include those that are of biological and physiological relevance to mammals and other organisms, including complex tissues, cells, hormones, steroids, lipids, proteins, peptides, metabolites, small molecules, ions (e.g., metal ions), viruses, polysaccharides, carbohydrates, biopolymers, synthetic polymers, and the like, and any combination thereof. Examples of specific analytes may include, but are not limited to, cortisol, aldosterone, testosterone, tobramycin, kanamycin, epidermal growth factor receptor, immunoglobin heavy chains, prostate-specific membrane antigen, melamine, milk allergens, protein tyrosine kinase, nucleolin, thrombin, vascular endothelial growth factor, vimentin, receptor binding domain of spike protein 1 for the COVID-19 virus, and the like, and any combination thereof.

The molecular beacons prepared as described herein may be provided as part of an analyte sensor, such as an electrochemical sensor. The molecular beacons may be tethered to an electrode, such as a working electrode, or in proximity to a working electrode. The tethering is not considered to be particularly limiting, provided that the molecular beacon(s) is capable of interacting with a target analyte of interest to structurally switch between its target-unbound and target-bound states, thereby permitting detection of the analyte, such as by detection of current, voltage, or other means, as described herein. For example, the molecular beacon may be tethered by hybridization, coating, adhesion, adsorption, other chemical or mechanical bonding, and the like, and any combination thereof. The electrode surface receiving the aptamer could also be prepared in any number of ways, such as by chemical functionalization, by using self-assembled monolayers or by other forms of physi- or chemi-sorption of a molecular scale interposing layer.

Accordingly, the present disclosure provides analyte sensors for tethering one or more molecular beacons as described herein to a substrate and/or electrode, in which the molecular beacon provides a signal that changes due to a target analyte being bound or unbound to the molecular beacon. As provided above, in one or all aspects, the molecular beacon signal may be electrochemical in nature and its incorporation in an electrochemical sensor comprises a working electrode and at least one additional electrode. Here, at least one additional electrode may comprise a working electrode, a counter-reference electrode, or separate counter and reference electrodes. That is, the analyte sensors comprising the molecular beacons of the present disclosure may be configured as a two-electrode, three-electrode configuration, or four-electrode configuration. When a reference electrode is omitted, the at least one additional electrode may serve as a counter-reference electrode.

The molecular beacon is positioned on or near the at least one working electrode and generate detectable electrical energy (current and/or voltage) that is correlative to the state of the molecular beacon being in either its analyte bound state or analyte unbound state, and thus used to determine the presence of the target analyte. That is, a difference in signal magnitude before and after exposing the molecular beacon to the analyte of interest may allow a concentration of the analyte to be determined. The difference signal may be correlated to an analyte concentration by consulting a lookup table, calibration curve, or the like. Optionally, the analyte sensors of the present disclosure may further comprise a processor configured to determine a concentration of the analyte based upon the change in magnitude of the signal.

### EXAMPLE EMBODIMENTS

### Embodiments of the Present Disclosure Include

Embodiment A: A method comprising: obtaining a nucleotide sequence for an aptamer that binds to a target analyte, the aptamer comprising a binding domain nucleotide sequence, a first domain nucleotide sequence, and a displacement domain nucleotide sequence complementary to the first domain nucleotide sequence; determining a first Gibbs free energy value for the aptamer; and developing a molecular beacon based on the nucleotide sequence of the aptamer by preserving the binding domain nucleotide sequence and truncating or extending one or both of the first domain nucleotide sequence or the displacement domain nucleotide sequence, the truncating or extending performed such that the molecular beacon comprises a second Gibbs free energy value that is greater than the first Gibbs free energy value of the aptamer.

Embodiment A may have one or more or all of the following additional elements in any combination:

Element A1: Wherein the developing is performed in silico.

Element A2: Further comprising determining a first apparent electron rate value (k1) representative of the molecular beacon being bound to the target analyte at the preserved binding domain nucleotide sequence and a second apparent electron rate value (k2) representative of the molecular beacon being unbound to the target analyte at the preserved binding domain nucleotide sequence, wherein the developing further comprises performing the truncating or extending such that the difference between the first apparent electron rate value and the second electron rate value is maximized.

Element A3: Further comprising determining a first apparent electron rate value (k1) representative of the molecular beacon being bound to the target analyte at the preserved binding domain nucleotide sequence and a second apparent electron rate value (k2) representative of the molecular beacon being unbound to the target analyte at the preserved binding domain nucleotide sequence, wherein the developing further comprises performing the truncating or extending such that the difference between the first apparent electron rate value and the second electron rate value is maximized, wherein the ratio of k1/k2 or k2/k1 is in the range of 2 to 1000.

Element A4: Further comprising determining a first apparent electron rate value (k1) representative of the molecular beacon being bound to the target analyte at the preserved binding domain nucleotide sequence and a second apparent electron rate value (k2) representative of the molecular beacon being unbound to the target analyte at the preserved binding domain nucleotide sequence, wherein the developing further comprises performing the truncating or extending such that the difference between the first apparent electron rate value and the second electron rate value is maximized, wherein the developing is performed in silico.

Element A5: Wherein the target analyte comprises at least one selected from the group consisting of a complex tissue, a cell, a hormone, a steroid, a lipid, a protein, a peptide, a metabolite, a small molecule, an ion, a virus, a polysaccharide, a carbohydrate, a biopolymer, a synthetic polymer, and any combination thereof.

Element A6: Wherein the target analyte comprises at least one selected from the group consisting of cortisol, aldosterone, testosterone, tobramycin, kanamycin, epidermal growth factor receptor, immunoglobin heavy chain, prostate-specific membrane antigen, melamine, milk allergens, protein tyrosine kinase, nucleolin, thrombin, vascular endothelial growth factor, vimentin, receptor binding domain of spike protein 1 for the COVID-19 virus, and any combination thereof.

Element A7: Wherein the target analyte is cortisol.

Element A8: Wherein the molecular beacon comprises a 5′ nucleotide end and a 3′ nucleotide end, and further comprising tethering one of the 5′ nucleotide end or the 3′ nucleotide end to a substrate.

Element A9: Wherein the molecular beacon comprises a 5′ nucleotide end and a 3′ nucleotide end, and further comprising tethering one of the 5′ nucleotide end or the 3′ nucleotide end to a substrate, and wherein the tethering comprises at least one selected from the group consisting of hybridization, coating, adhesion, adsorption, chemical bonding, mechanical bonding, and any combination thereof.

Element A10: Wherein the molecular beacon comprises a 5′ nucleotide end and a 3′ nucleotide end, and further comprising tethering one of the 5′ nucleotide end or the 3′ nucleotide end to a substrate, and wherein the substrate is a working electrode.

Element A11: Wherein the molecular beacon comprises a 5′ nucleotide end and a 3′ nucleotide end, and further comprising tethering one of the 5′ nucleotide end or the 3′ nucleotide end to a substrate, and further comprising functionalizing one of the 5′ nucleotide end or the 3′ nucleotide end that is not tethered to the substrate with a redox active center.

Element A12: Wherein the molecular beacon comprises a 5′ nucleotide end and a 3′ nucleotide end, and further comprising tethering one of the 5′ nucleotide end or the 3′ nucleotide end to a substrate, and further comprising functionalizing one of the 5′ nucleotide end or the 3′ nucleotide end that is not tethered to the substrate with a redox active center, and wherein the redox active center comprises at least one selected from the group consisting of a metallocene, methylene blue, an anthraquinone, a benzoquinone, a napthoquinone, a viologen, nile blue, any derivatives thereof, and any combination thereof.

By way of non-limiting example, exemplary combinations applicable to Embodiment A include: A1 in combination with A2, A3, A5, A6, A7, and/or A8; A2 in combination with A3, A4, and/or any of A5 through A8; A9 in combination with A10, A11, A12 and/or any of A1 through A8; or any of A1 through A12 in any combination.

Embodiment B: A molecular beacon comprising: a portion of a nucleotide sequence of an aptamer that binds to a target analyte and has a first Gibbs free energy value, the portion of the nucleotide sequence forming the molecular beacon comprising: a preserved binding domain nucleotide sequence of the aptamer, wherein the binding domain sequence of the aptamer binds to the target analyte; and a truncated or extended one or both of a first domain nucleotide sequence of the aptamer or a displacement domain nucleotide sequence of the aptamer, wherein the truncation or extension is performed to develop the molecular beacon comprising a second Gibbs free energy value that is greater than the first Gibbs free energy value of the aptamer.

Embodiment B may have one or more or all of the following additional elements in any combination:

Element B1: Wherein the truncation or extension is performed in silico.

Element B2: Wherein the truncation or extension is further performed to develop the molecular beacon having a first apparent electron rate value (k1) representative of the molecular beacon being bound to the target analyte at the preserved binding domain nucleotide sequence and a second apparent electron rate value (k2) representative of the molecular beacon being unbound to the target analyte at the preserved binding domain nucleotide sequence, wherein that the difference between the first apparent electron rate value and the second electron rate value is maximized.

Element B3: Wherein the truncation or extension is further performed to develop the molecular beacon having a first apparent electron rate value (k1) representative of the molecular beacon being bound to the target analyte at the preserved binding domain nucleotide sequence and a second apparent electron rate value (k2) representative of the molecular beacon being unbound to the target analyte at the preserved binding domain nucleotide sequence, wherein that the difference between the first apparent electron rate value and the second electron rate value is maximized, and wherein the ratio of k1/k2 or k2/k1 is in the range of 2 to 1000.

Element B4: Wherein the truncation or extension is further performed to develop the molecular beacon having a first apparent electron rate value (k1) representative of the molecular beacon being bound to the target analyte at the preserved binding domain nucleotide sequence and a second apparent electron rate value (k2) representative of the molecular beacon being unbound to the target analyte at the preserved binding domain nucleotide sequence, wherein that the difference between the first apparent electron rate value and the second electron rate value is maximized, and wherein the truncation or extension is performed in silico.

Element B5: Wherein the portion of the nucleotide sequence forming the molecular beacon is SEQ ID No. 1.

Element B6: Wherein the portion of the nucleotide sequence forming the molecular beacon comprises a 5′ nucleotide end and a 3′ nucleotide end, and wherein one of the 5′ nucleotide end or the 3′ nucleotide end is functionalized with a redox active center.

By way of non-limiting example, exemplary combinations applicable to Embodiment B include: B1 in combination with B2, B5 and/or B6; B2 in combination with B3, B4, B5 and/or B6; or any of B1 through B6 in any combination.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the incarnations of the present inventions. At the very least, and not as an attempt to limit the application of the doctrine of equivalents to the scope of the claim, each numerical parameter should at least be construed in light of the number of reported significant digits and by applying ordinary rounding techniques.

One or more illustrative incarnations incorporating one or more invention elements are presented herein. Not all features of a physical implementation are described or shown in this application for the sake of clarity. It is understood that in the development of a physical embodiment incorporating one or more elements of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps.

Therefore, the present invention is well adapted to attain the ends and advantages mentioned as well as those that are inherent therein. The particular examples and configurations disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative examples disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. A11 numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

The invention claimed is:

1. A method comprising:
obtaining a nucleotide sequence for an aptamer that binds to a target analyte, the aptamer comprising a binding domain nucleotide sequence, a first domain nucleotide sequence, and a displacement domain nucleotide sequence complementary to the first domain nucleotide sequence;
determining a first Gibbs free energy value for the aptamer; and
developing a molecular beacon based on the nucleotide sequence of the aptamer by preserving the binding domain nucleotide sequence and truncating or extending one or both of the first domain nucleotide sequence or the displacement domain nucleotide sequence, the truncating or extending performed such that the molecular beacon comprises a second Gibbs free energy value that is greater than the first Gibbs free energy value of the aptamer.

2. The method of claim 1, wherein the developing is performed in silico.

3. The method of claim 1, further comprising determining a first apparent electron rate value (k₁) representative of the molecular beacon being bound to the target analyte at the preserved binding domain nucleotide sequence and a second apparent electron rate value (k₂) representative of the molecular beacon being unbound to the target analyte at the preserved binding domain nucleotide sequence, wherein the developing further comprises performing the truncating or extending such that the difference between the first apparent electron rate value and the second electron rate value is maximized.

4. The method of claim 3, wherein the ratio of k₁/k₂ or k₂/k₁ is in the range of 2 to 1000.

5. The method of claim 3, wherein the developing is performed in silico.

6. The method of claim 1, wherein the target analyte comprises at least one selected from the group consisting of a complex tissue, a cell, a hormone, a steroid, a lipid, a protein, a peptide, a metabolite, a small molecule, an ion, a virus, a polysaccharide, a carbohydrate, a biopolymer, a synthetic polymer, and any combination thereof.

7. The method of claim 1, wherein the target analyte comprises at least one selected from the group consisting of cortisol, aldosterone, testosterone, tobramycin, kanamycin, epidermal growth factor receptor, immunoglobin heavy chain, prostate-specific membrane antigen, melamine, milk allergens, protein tyrosine kinase, nucleolin, thrombin, vascular endothelial growth factor, vimentin, receptor binding domain of spike protein 1 for the COVID-19 virus, and any combination thereof.

8. The method of claim 1, wherein the target analyte is cortisol.

9. The method of claim 1, wherein the molecular beacon comprises a 5′ nucleotide end and a 3′ nucleotide end, and further comprising tethering one of the ′5 nucleotide end or the 3′ nucleotide end to a substrate.

10. The method of claim 9, wherein the tethering comprises at least one selected from the group consisting of hybridization, coating, adhesion, adsorption, chemical bonding, mechanical bonding, and any combination thereof.

11. The method of claim 9, wherein the substrate is a working electrode.

12. The method of claim 9, further comprising functionalizing one of the 5′ nucleotide end or the 3′ nucleotide end that is not tethered to the substrate with a redox active center.

13. The method of claim 12, wherein the redox active center comprises at least one selected from the group consisting of a metallocene, methylene blue, an anthraquinone, a benzoquinone, a napthoquinone, a viologen, nile blue, any derivatives thereof, and any combination thereof.

14. A molecular beacon comprising:
a portion of a nucleotide sequence of an aptamer that binds to a target analyte and has a first Gibbs free energy value, the portion of the nucleotide sequence forming the molecular beacon comprising:
a preserved binding domain nucleotide sequence of the aptamer, wherein the binding domain sequence of the aptamer binds to the target analyte; and
a truncated or extended one or both of a first domain nucleotide sequence of the aptamer or a displacement domain nucleotide sequence of the aptamer, wherein the truncation or extension is performed to develop the molecular beacon comprising a second Gibbs free energy value that is greater than the first Gibbs free energy value of the aptamer.

15. The molecular beacon of claim 14, wherein the truncation or extension is performed in silico.

16. The molecular beacon of claim 14, wherein the truncation or extension is further performed to develop the molecular beacon having a first apparent electron rate value (k₁) representative of the molecular beacon being bound to the target analyte at the preserved binding domain nucleotide sequence and a second apparent electron rate value (k₂) representative of the molecular beacon being unbound to the target analyte at the preserved binding domain nucleotide sequence, wherein that the difference between the first apparent electron rate value and the second electron rate value is maximized.

17. The molecular beacon of claim 16, wherein the ratio of k₁/k₂ or k₂/k₁ is in the range of 2 to 1000.

18. The molecular beacon of claim 16, wherein the truncation or extension is performed in silico.

19. The molecular beacon of claim 14, wherein the portion of the nucleotide sequence forming the molecular beacon is SEQ ID No. 1.

20. The molecular beacon of claim 14, wherein the portion of the nucleotide sequence forming the molecular beacon comprises a 5′ nucleotide end and a 3′ nucleotide end, and wherein one of the ′5 nucleotide end or the 3′ nucleotide end is functionalized with a redox active center.


SEQUENCE LISTING (1 sequence)

SEQ ID NO: 1 - DNA, 46 bp, Artificial sequence
  Single-stranded DNA sequence
ctctcgggacgacgcccgcatgttccatggatagtcttgactagtc